SEQUENCE LISTING (29 sequences)

SEQ ID NO: 1 - DNA, 22 bp, Actinobacillus actinomycetemcomitans
caggtaagtacttgtacttatg

SEQ ID NO: 2 - DNA, 17 bp, Actinobacillus actinomycetemcomitans
ggattggggtttagccc

SEQ ID NO: 3 - DNA, 21 bp, Actinobacillus actinomycetemcomitans
aggagaaagcttgctttcttg

SEQ ID NO: 4 - DNA, 20 bp, Actinobacillus actinomycetemcomitans
ggataagggttgcgctcgtt

SEQ ID NO: 5 - DNA, 21 bp, Porphyromonas gingivalis
catgatcttagcttgctaagg

SEQ ID NO: 6 - DNA, 22 bp, Prevotella intermedia
cattatgtgcttgcacattctg

SEQ ID NO: 7 - DNA, 20 bp, Bacteroides forsythus
gakggtagcaatacctgtcg

SEQ ID NO: 8 - DNA, 20 bp, Treponema denticola
cggtaagggagagcttgctc

SEQ ID NO: 9 - DNA, 20 bp, Treponema denticola
taagggagagcttgctctcc

SEQ ID NO: 10 - DNA, 18 bp, Treponema denticola
gcaagtcgaacggtaagg

SEQ ID NO: 11 - DNA, 19 bp, Unknown
  Oligonucleotide derived from periodontitis/caries associated bacterium
ctgctgcctcccgtaggag

SEQ ID NO: 12 - DNA, 19 bp, Streptococcus mutans
actgtgcttgcacaccgtg

SEQ ID NO: 13 - DNA, 19 bp, Streptococcus sobrinus
caccggacttgctccagtg

SEQ ID NO: 14 - DNA, 18 bp, Actinobacillus actinomycetemcomitans
ggattggggtttagcccc

SEQ ID NO: 15 - DNA, 15 bp, Actinobacillus actinomycetemcomitans
cgaagaagaactcag

SEQ ID NO: 16 - DNA, 17 bp, Actinobacillus actinomycetemcomitans
tccgaagaagaactcag

SEQ ID NO: 17 - DNA, 16 bp, Actinobacillus actinomycetemcomitans
cgcgtagaatcgggag

SEQ ID NO: 18 - DNA, 16 bp, Actinobacillus actinomycetemcomitans
cgcgtagggtcgggag

SEQ ID NO: 19 - DNA, 16 bp, Actinobacillus actinomycetemcomitans
cgcgtagagtcgggag

SEQ ID NO: 20 - DNA, 17 bp, Porphyromonas gingivalis
catacacttgtattatt

SEQ ID NO: 21 - DNA, 19 bp, Porphyromonas gingivalis
catacacttgtattattgc

SEQ ID NO: 22 - DNA, 17 bp, Prevotella intermedia
atgttgtccacatatgg

SEQ ID NO: 23 - DNA, 15 bp, Bacteroides forsythus
aacaggggttccgca

SEQ ID NO: 24 - DNA, 20 bp, Treponema denticola
cattyacctttatgtaaatg

SEQ ID NO: 25 - DNA, 20 bp, Treponema denticola
catttacataaaggtraatg

SEQ ID NO: 26 - DNA, 16 bp, Treponema denticola
tgggtgacctgccctg

SEQ ID NO: 27 - DNA, 15 bp, Treponema denticola
cttyaggatgggccc

SEQ ID NO: 28 - DNA, 19 bp, Streptococcus sobrinus
cataagaggagttaactca

SEQ ID NO: 29 - DNA, 17 bp, Streptococcus mutans
ccgcataatattaatta

(19) United States
(12) Patent Application Publication
(10) Pub. No.: US 2005/0014154 A1
(43) Pub. Date: Jan. 20, 2005

(54) Method in the form of a dry rapid test for detecting nucleic acids

(76) Inventor: Michael Weizenegger, Wiesloch (DE)

Correspondence Address: SHANKS & HERBERT, 1301 K STREET, N.W., SUITE 1100 - EAST TOWER, WASHINGTON, DC, 20005, US

(21) Appl. No.: 10/494,661

(22) PCT Filed: Nov. 5, 2002
(86) PCT No.: PCT/EP02/12333

(30) Foreign Application Priority Data
Nov. 5, 2001 (DE) 101 54 291.7

(51) Int. Cl. C12Q 1/68

(52) U.S. Cl. 435/6

(57) ABSTRACT

The invention relates to the field of diagnostics of nucleic acids, especially to a highly sensitive method for detecting, differentiating and characterizing nucleic acids in the form of a dry rapid test. The inventive dry rapid test contains a chromatographic material that comprises a sample reception zone, a separation zone including a binding area, in which one or more sequence-specific nucleic acid probes are immobilized, and a zone which absorbs a liquid down-stream of the separation zone including a binding area. According to the inventive method, the sequence-specific nucleic acid probes are immobilized via a polymer linker. The method comprises the following steps: i) denaturing, in the case of double-stranded nucleic acids, the nucleic acid to be detected and then neutralizing it, ii) applying the nucleic acid to be detected to the sample reception zone in a run buffer which contains mildly denaturing agents, iii) the nucleic acid moving from the sample reception zone in direction of the liquid-absorbing zone, (iv) contacting the nucleic add to be detected in the binding area of the separation path with the sequence-specific nucleic acid probe and hybridizing it with the sequence-specific nucleic acid probe, v) detecting the nucleic acid or the hybridization of the nucleic acid with the sequence-specific nucleic acid probe via a label that is attached to the nucleic acid to be detected or via the detection of a label of the nucleic acid double strand. The invention further relates to a device for carrying out the method according to the invention.

The present invention relates to the field of diagnosis of nucleic acids. More specifically, the present invention relates to a highly sensitive method for detecting, differentiating and characterizing nucleic acids in the form of a rapid dry assay; said rapid dry assay containing a chromatographic material comprising
a sample-receiving zone,
a separating zone having a binding region in which one or more sequence-specific nucleic acid probes are immobilized and
a liquid-absorbing zone downstream of said separating zone having a binding region, characterized in that said sequence-specific nucleic acid probes are immobilized via a polymeric linker, and further characterized in that said method comprises the following steps:
i) in the case of double-stranded nucleic acids, denaturing and then neutralizing the nucleic acid to be detected,
ii) applying the nucleic acid to be detected to said sample-receiving zone in a running buffer containing mildly denaturing agents,
iii) the nucleic acid to be detected moving from said sample-receiving zone in the direction of said liquid-absorbing zone,
iv) the nucleic acid to be detected being contacted with, and hybridizing to, said sequence-specific nucleic acid probe in said binding region of the separating section,
v) detecting the nucleic acid to be detected or hybridization of the nucleic acid to be detected to said sequence-specific nucleic acid probe via a label attached to the nucleic acid to be detected or via labeling of the nucleic acid double strand. Thus, for example, detection during an antibody conjugate against a DNA double strand or a DNA/RNA double strand (the latter, for example in the case that the probe or the target sequence is RNA) is also regarded as labeling. The present invention further relates to an apparatus for carrying out the method of the invention.

The detection of nucleic acids by probes is described in the prior art.

Methods for rapid and simple detection of nucleic acids become more and more important in the areas of medicine, environment, food and forensics. Owing to the high specificity and sensitivity of nucleic acid-based methods, these assays play an important part in identifying and differentiating pathogens, contaminating organisms or else in subtyping bacteria or viruses and studying genetic polymorphisms.

If only small amounts of the nucleic acid to be detected are present in the sample, said nucleic acid to be detected is amplified. Various reactions may be used as nucleic acid amplification reaction. Preference is given to using the polymerase chain reaction (PCR). The various embodiments of the PCR technique are known to the skilled worker, see, for example, Mullis (1990) Target amplification for DNA analysis by the polymerase chain reaction. Ann. Biol Chem (Paris) 48(8), 579-582. Further amplification techniques which may be applied are nucleic acid strand-based amplification (NASBA), transcriptase mediated amplification (TMA), reverse transcriptase polymerase chain reaction (RT-PCR), Q-β replicase amplification (β-Q-Replicase) and the single strand displacement amplification (SDA). NASBA and other transcription-based amplification methods are discussed in Chan and Fox, Reviews in Medical Microbiology (1999), 10 (4), 185-196.

The simplest form of detecting the nucleic acid to be detected comprises cutting the amplicon specifically, for example by digestion with a restriction enzyme, and analyzing the ethidium bromide-stained fragments produced on an agarose gel. Hybridization systems are also very common. The hybridization is normally carried out by immobilizing either the composition containing the amplification product or a part thereof or the probe on a solid phase and contacting it with the in each case other hybridization partner. Possible solid phases are a large variety of materials, for example nylon, nitrocellulose, polystyrene, silicatic materials, etc. It is also conceivable to use a microtiter plate as solid phase. This may also involve the target sequence hybridizing with a capture probe in solution beforehand and then binding said capture probe to a solid phase.

Usually, amplification of the nucleic acid to be detected involves at least one labeled probe or at least one labeled primer. A large variety of labels is possible here, such as fluorescent dyes, biotin or digoxigenin, for example. Known fluorescent labels are fluorescein, FITC (fluoroisothiocyanate), cyanine dyes, etc. The labels are normally covalently linked to the oligonucleotides. While a fluorescent label can be detected directly, biotin and digoxigenin labels may be detected after incubation with suitable binding molecules. For example, a biotin-labeled oligonucleotide may be detected by contacting it with a solution containing streptavidin coupled to an enzyme, said enzyme, for example peroxidase or alkaline phosphatase, converting a substrate which produces a dye or results in chemiluminescence.

All of these methods are labor-intensive and generally require several hours. Automation of said methods may drastically reduce manual labor and analysis time, but requires equipment whose purchasing and running costs are high and which can be used only with large numbers of samples and in specialized laboratories. This stands in the way especially of the requirements of “point of care” diagnostics in which nucleic acid-based diagnosis is intended to be carried out also as individual detection rapidly and, if possible, without any personnel specially trained in the nucleic acid techniques.

U.S. Pat. No. 6,037,127 describes a simple method for detecting non-denatured nucleic acids. This assay is carried out in the form of a rapid dry assay in which one embodiment comprises immobilizing nucleic acid probes on the chromatographic material of the test strip. The assay methods described here are not highly sensitive, however.

It was therefore the object of the present invention to develop a highly sensitive method for detecting nucleic acids, which as a rapid assay is easily to be carried out and which makes possible sequence-specific identification, differentiation and characterization of nucleic acids.

According to the invention, this object was achieved by a highly sensitive method for detecting, differentiating and characterizing nucleic acids in the form of a rapid dry assay; said rapid dry assay containing a chromatographic material comprising
a sample-receiving zone,
a separating zone having a binding region in which one or more sequence-specific nucleic acid probes are immobilized and
a liquid-absorbing zone downstream of said separating zone having a binding region, characterized in that said sequence-specific nucleic acid probes are immobilized via a polymeric linker, and further characterized in that said method comprises the following steps:
i) in the case of double-stranded nucleic acids, denaturing and then neutralizing the nucleic acid to be detected,
ii) applying the nucleic acid to be detected to said sample-receiving zone in a running buffer containing mildly denaturing agents,
iii) the nucleic acid to be detected moving from said sample-receiving zone in the direction of said liquid-absorbing zone,
iv) the nucleic acid to be detected being contacted with, and hybridizing to, said sequence-specific nucleic acid probe in said binding region of the separating section,
v) detecting the nucleic acid to be detected or hybridization of the nucleic acid to be detected to said sequence-specific nucleic acid probe via a label attached to the nucleic acid to be detected or via labeling of the nucleic acid double strand. Thus, for example, detection during an antibody conjugate against a DNA double strand or a DNA/RNA double strand (the latter, for example in the case that the probe or the target sequence is RNA) is also regarded as labeling.

The term nucleic acid and oligonucleotide means in accordance with the present invention primers, samples, probes and oligomeric fragments which are detected. The term nucleic acid and oligonucleotide is furthermore generic for polydeoxyribonucleotides (comprising 2-deoxy-D-ribose) and for polyribonucleotides (comprising D-ribose) or for any other type of polynucleotide which is an N-glycoside of a purine base or of a pyrimidine base, or of a modified purine base or modified pyrimidine base. Included are thus also according to the invention PNAs, i.e. polyamides having purine/pyrimidine bases. In accordance with the present invention, the terms nucleic acid and oligonucleotide are not regarded as being different; more specifically, use of said terms is not intended to implicate any distinction with respect to length. Said terms include both double- and single-stranded DNA and double- and single-stranded RNA.

Rapid dry assay means in accordance with the present invention an apparatus enabling the product to be analyzed to be chromatographically fractionated. In accordance with the present invention, particular preference is given to using a chromatographic material having a pore size of 4 μm or more, most preferably of more than 8 μm. The analyte is transported by capillary forces of said chromatographic material to the reaction zones such as the separating zone, for example.

Since the analyte is mainly hydrophilic, hydrophilic properties of said chromatographic material of the test strip are important for carrying out the method of the invention. Said chromatographic material may comprise inorganic powders such as silicatic materials, magnesium sulfate and aluminum, and may furthermore comprise synthetic or modified naturally occurring polymers such as nitrocellulose, cellulose acetate, cellulose, polyvinyl chloride or polyvinyl acetate, polyacrylamide, nylon, crosslinked dextran, agarose, polyacrylate, etc., and may furthermore comprise coated materials such as ceramic materials and glass. Most preference is given to using nitrocellulose as chromatographic material. In addition, the introduction of positively charged ionic groups into nitrocellulose or nylon membranes, for example, may improve the hydrophilic properties of said chromatographic material.

The rapid dry assay comprises a sample-receiving zone, a separating zone having a binding region in which one or more sequence-specific nucleic acid probes are immobilized and a liquid-absorbing zone downstream of said separating zone having a binding region. The rapid dry assay may have a plurality of separating zones. The rapid dry assay may additionally comprise zones containing labeling substances which bind to the analyte when the latter passes through this zone. A typical example is a zone containing a gold conjugate for labeling the passing analyte. The rapid dry assay may have in particular the form of a test strip.

Further commonly used variants relating to the rapid dry assay and to the chromatographic material are described in the prior art, in particular in U.S. Pat. No. 6,103,127.

The chromatographic material may be installed in a housing or the like. Said housing is usually water-insoluble, rigid and may comprise a multiplicity of organic and inorganic materials. It is important that the housing does not interfere with the capillary properties of the chromatographic material, that said holding does not bind test components unspecifically and that said housing does not interfere with the detection system.

Preference is given according to the invention to the polymeric linker or the polymeric linker with anchoring molecule being more than 30 nm in length.

The length of the linker is of crucial importance, in order to obtain high sensitivity of the immobilized probe. The linker acts as a spacer between probe and membrane. In the present case, said linkers are usually polymers which extend the target sequence-complementary part of the probe at the 5′ or 3′ end but which themselves are noncoding. They may be base sequences of a noncoding nucleic acid structure or other polymeric units such as, for example, polyethers, polyesters, and the like. The nature of said linker must be such that the latter is not or only weakly adversely influenced the hybridization properties of the probe. This may be avoided by the absence of self-complementary structures. The chemical preconditions for irreversible coupling of the probe to the support material must also be present. A crucial requirement for proper functioning of the probe, in addition to its properties of forming a stable hybrid with the target sequence, is the chemistry of coupling to the surface. Chemical groups must be present which make irreversible binding possible with the immobilization techniques used. Said groups may be amine groups, thiol groups, carbamides, succinimides, and the like.

Preference is given to the polymeric linker or the polymeric linker with anchoring molecule being more than 30 nm in length, particularly preferably more than 40 nm. According to the invention, preference is furthermore given to the linker being polythymidine.

The length of synthetic linkers is often limited. After a number of steps in chemical synthesis of oligonucleotides as linkers according to the phosphoramidite method, for example, yield and product quality are reduced to such a large extent that the oligonucleotide length is limited to approx. 100 monomers. Depending on the monomer, this corresponds approximately to a length of approx. 30-40 nm. Enzymatic methods of oligonucleotide extension, for example by a terminal transferase, always result in a mixture of long (up to several hundred monomers) and very short oligonucleotides.

The sequence-specific nucleic acid probes are immobilized to the membrane or to the chromatographic material preferably via a polymeric linker connected to an anchoring molecule. The most preferred anchoring molecule is psoralen. Psoralen, as a crosslinking reagent, provides the possibility of forming very long linkers from fully synthetic oligonucleotides. Psoralen is a polycyclic compound capable of coupling photochemically to pyrimidine residues with UV light of a wavelength of approx. 360 nm. The reaction with thymidine residues is particularly good. Coupling of probes having, for example, a psoralen-containing polythymidine extension at the 5′ end may produce extensions of the spacers or linkers by means of crosslinking of the molecules due to UV light action. A mixture of pure polythymidine without anchoring molecule and polythymidine with psoralen modification as anchoring molecule at the end of the polythymidine linker may be used to construct network structures which prove advantageous for the hybridization efficiency and sensitivity of the probes. Furthermore, it is possible to mix and photocrosslink the DNA with psoralen-labeled oligonucleotides. This improves adhesion of the probe to the surface. According to the invention, the anchoring molecule used may also be other molecules such as, for example: psoralen derivatives such as bis(PIP) Cn-psoralen, for example, or other photoreactive crosslinking and labeling reagents known to the skilled worker, such as, for example, simple aryl-azide crosslinkers, fluorinated aryl-azide crosslinkers or benzophenone-based crosslinkers.

However, other possibilities of generating spacers or linkers between the probe and the membrane are also known to the skilled worker. Probe oligonucleotides may be bound, for example, via proteins to the membrane surface. The proteins charged with the probe may then be bound to the porous membrane according to standard methods. An example of standard methods is coupling via homobifunctional coupling reagents or heterobifunctional coupling reagents. Homobifunctional ones have identical reactive groups. These are typically amines and/or thiols. Thiols may be coupled synthetically directly to oligonucleotides and may react with cysteine residues, for example, under oxidative conditions to give disulfide bridges. For an amine-amine coupling, amines may be coupled as homobifunctional coupling reagents synthetically directly to oligonucleotides and bound via imidoesters or succinimide esters to the surface or the protein.

Heterobifunctional coupling reagents have different reactive groups and allow coupling of various functional groups. Preference is given to the formation of amino-thiol couplings. A heterobifunctional coupling reagent which comprises both a succinimide ester maleimide or iodoacetamide may be used to couple thiolated oligonucleotides. Another important coupling agent is carbodiimides which couple carbonyl radicals to amines. The most important representative here is 1-ethyl-3-(3-dimethylaminopropyl)carbodiimide (EDAC). Here it is possible to couple amino-modified oligonucleotides to membranes containing carbonyl radicals. In this chemistry, the coupling reagent is not incorporated into the compound.

According to the invention, the nucleic acid probes are immobilized on the solid phase and said solid phase is then contacted with the composition containing the labeled nucleic acids to be detected or part thereof. Preferably at least two probes, more preferably at least five probes, still more preferably at least ten probes, are immobilized on the solid phase. Various probes may be immobilized in various zones.

According to the methods of the invention, the nucleic acid to be detected is labeled. A large variety of labels is possible here, such as fluorescent dyes, biotin or digoxigenin, for example. Known fluorescent labels are fluorescein, FITC, cyanine dyes, rhodamines, Rhodamin₆₀₀R phycoerythrin, Texas Red, etc.

A radiolabel such as, for example, ¹²⁵I, ³⁵S, ³²P, ³⁵P is also conceivable.

Particle labeling, for example, with latex, is also conceivable. Such particles are usually dry, in the micron range and uniform.

The labels are normally covalently linked to the oligonucleotides. While a fluorescent label, for example, can be detected directly, biotin and digoxigenin labels may be detected after incubation with suitable binding molecules or conjugate partners.

Examples of binding partners other than biotin/streptavidin are antigen/antibody systems, hapten/anti-hapten systems, biotin/avidin, folic acid/folate-binding proteins, complementary nucleic acids, proteins A, G and immunoglobulin, etc. (M. N. Bobrov, et al. J. Immunol. Methods, 125, 279, (1989)).

For example, it is possible to detect a biotin-labeled oligonucleotide by contacting it with a solution containing streptavidin coupled to an enzyme, said enzyme, for example, peroxidase or alkaline phosphatase, converting a substrate which produces a dye or results in chemiluminescence. Possible enzymes for this use purpose are hydrolases, lyases, oxido reductases, transferases, isomerases and ligases. Further examples are peroxidases, glucose oxidases, phosphatases, esterases and glycosidases. Methods of this kind are known per se to the skilled worker (Wetmur JG, Crit Rev Biochem Mol Biol 1991; (3-4): 227-59; Temsamani J. et al. Mol Biotechnol 1996 Jun; 5(3): 223-32). In some methods in which enzymes act as conjugate partners color-changing substances must be present (Tijssen, P. Practice and Theory of Enzyme Immunoassays in Laboratory Techniques in Biochemistry and Molecular Biology, Edited by R. H. Burton and P. H. van Knippenberg (1998)).

Another preferred conjugate comprises an enzyme which is coupled to an antibody (Williams, J. Immunol. Methods, 79, 261 (1984)). It is furthermore common to label the nucleic acid to be detected with a gold-streptavidin conjugate, enabling a biotin-labeled target nucleotide to be detected. However, binding partners forming covalent bonds with one another, such as, for example, sulfhydryl-reactive groups such as maleimides and haloacetyl derivatives and amine-reactive groups such as isothiocyanates, succinimidyl esters and sulfonyl halides, are also conceivable.

In the case of labeling with conjugates, the detectable conjugate may be applied to a zone of the rapid dry assay through which the nucleic acid to be detected passes, with the conjugate partner having been introduced into said nucleic acid to be detected.

If the nucleic acids to be detected are labeled, then the probes are usually unlabeled. The nucleic acids to be detected are thus labeled essentially according to methods described in the prior art (U.S. Pat. No. 6,037,127).

The label may be introduced into the nucleic acid to be detected by chemical or enzymic methods or by direct incorporation of labeled bases into said nucleic acid to be detected. In a preferred embodiment, sequences to be detected, which have incorporated labels, are produced by means of labeled bases or labeled primers during PCR. Labeled primers may be prepared by chemical synthesis, for example by means of the phosphoramidite method by substituting labeled phosphoramidite bases for bases of said primer during primer synthesis. As an alternative to this, it is possible to prepare primers containing modified bases to which labels are chemically bound after primer synthesis.

Methods without amplifying the nucleic acid to be detected and/or providing it with a modification are also possible. For example, ribosomal RNA species can specifically hybridize with a DNA probe and be detected as RNA/DNA hybrid, using an RNA/DNA-specific antibody. Another possibility is introducing labels with the aid of T4 polynucleotide kinase or of a terminal transferase enzyme. Thus it is conceivable to introduce radioactive or fluorescent labels (Sambrook et al., Molecular Cloning, Cold Spring Harbor Laboratory Press, Vol. 2, 9.34-9.37 (1989); Cardullo et al. PNAS, 85, 8790; Morrison, Anal. Biochem, 174, 101 (1988).

Labels may be introduced at one or both ends of the nucleic acid sequence of the nucleic acid to be detected. Labels may also be introduced within the nucleic acid sequence of the nucleic acid to be detected. It is also possible to introduce a plurality of labels into a nucleic acid to be detected.

According to the invention, preference is given to carrying out denaturation according to method step i) with NaOH and neutralization according to method step i) with a phosphate buffer or Tris buffer. Denaturation may also be achieved by other measures such as, for example, boiling at a temperature higher than 95° C., possibly with the addition of mildly denaturing chemicals. Denaturation of the nucleic acid to be detected is always required, if double-stranded nucleic acids are present in the sample. According to the invention, preference is furthermore given to the running buffer according to method step ii) being phosphate or TRIS buffer and containing one or more of the following as mildly denaturing agent: formamide, DMSO, urea. However, it is also possible according to the invention to use any running buffer listed below for neutralization and as running buffer. The buffer used for neutralization may be identical to the running buffer.

Buffers and solvents which may be used for the method of the invention are known and examples are described in U.S. Pat. No. 4,740,468 and U.S. Pat. No. 6,037,127. The running buffer pH is usually in the range of 4-11, preferably in the range of 5-11 and most preferably in the range of 6-9.

The pH is chosen so as to retain a considerable degree of binding affinity between all binding terminals, including the hybridizing nucleic acids, and also to be able to obtain an optimal signal from the signal-producing system. Buffers which are typically used contain borate, phosphate, carbonate, Tris, barbital, and the like. The choice of a suitable buffer is normally not critical for the method of the invention. However, some buffers may be more suitable than others for special assays. Conventional hybridization methods are carried out using heated solutions in hybridization ovens or water baths, typically at from 30 to 70° C., preferably at 50° C. A nucleic acid detection system in rapid dry assay format must be operated at room temperature. In order to obtain good sensitivities, it is therefore necessary to add the abovementioned mildly denaturing agents to the running buffer. The method of the invention may, of course, be carried out at any temperature between 4° C. and 50° C. However, the most convenient temperature is room temperature which is therefore preferred.

The term hybridization refers to the formation of duplex structures by two single-stranded nucleic acids, owing to complementary base pairing. Hybridization can take place between complementary nucleic acid strands or between nucleic acid strands which have relatively small mismatched regions. The stability of said nucleic acid duplex is measured by way of the melting temperature Tₘ. The melting temperature Tₘ is the temperature (with defined ionic strength and pH) at which 50% of base pairs are dissociated.

Conditions under which merely fully complementary nucleic acids hybridize are referred to as stringent hybridization conditions. Stringent hybridization conditions are known to the skilled worker (e.g. Sambrook et al., 1085, Molecular Cloning—A Laboratory Manual, Cold Spring Harbor Laboratory, Cold Spring Harbor, N.Y.). In general, stringent conditions are selected so as for the melting temperature to be 5° C. lower than the Tₘ for the specific sequence at defined ionic strength and pH. If said hybridization is carried out under less stringent conditions, sequence mismatches are tolerated. It is possible to control the degree of sequence mismatches by altering the hybridization conditions.

Carrying out the hybridization under stringent conditions is particularly important for the method of the invention. Stringent means in accordance with the present invention that the detection method allows for unambiguous distinction between a positive reaction and a negative reaction in the reaction field of the strip. This may be achieved in particular by the following measures:

Probe structure: via the length of the target sequence-complementary structure of the probe; preference is given to 15 to 20mers.

Running buffer: the salt content influences stringency.

The ionic strength is preferably between 100-400 mM, particularly preferably at 250 mM.

It is furthermore possible to individually adjust and optimize the stringency by the abovementioned mildly denaturing substances in the running buffer (DMSO, formamide, urea). The stringency is also influenced by the running buffer pH. All of the measures mentioned above are ultimately measures which influence hydrogen bonds.

According to the invention, preference is given to the sequence-specific nucleic acid probe being a nucleic acid having a specific sequence which hybridizes to the nucleic acid to be detected under stringent hybridization conditions. Specific sequence means a defined nucleic acid structure distinguishable from a multiplicity of other structures. This may be for differentiating microorganisms and viruses or else for distinguishing nucleic acid polymorphisms in genetic or epidemiological problems.

The nucleic acid to be detected may be isolated from a multiplicity of organisms such as bacterial and viral pathogens. The nucleic acid to be detected may also be the subject of a diagnostic test for a genetically caused disease. The nucleic acid to be detected may be from any conceivable source, if it is intended to detect, differentiate or characterize detection of the nucleic acid or parts thereof. Most of the time, the nucleic acid to be detected is not detected directly but must be amplified beforehand (for a description of possible amplification methods, see also U.S. Pat. No. 6,037,127).

The below-described embodiment of the method of the invention is intended to illustrate the invention in more detail. In this embodiment, the nucleic acid to be detected is a fragment from the genome of a parodontitis-associated bacterium or is complementary thereto. In said embodiment, the sequence-specific nucleic acid probe is a nucleic acid having a species-specific sequence which hybridizes to the nucleic acid to be detected under stringent hybridization conditions. The sequence-specific nucleic acid probe is preferably selected from any of the following sequences or is complementary to said sequences: SEQ ID No. 1-29, or is a fragment thereof (see also FIGS. 6 and 7).

The skilled worker appreciates that it is possible, starting from the teaching of the present invention, also to design probes which slightly deviate from the probes of the invention but which nevertheless function. Conceivable probes are thus also those which, compared to the probes of the invention having the sequences SEQ ID No. 1-29, are extended or truncated by at least one, two or three nucleotides at the 5′ and/or 3′ end. It is likewise conceivable that individual or a few nucleotides of a probe can be replaced with other nucleotides, as long as the specificity of said probe and the melting point of said probe are not altered too much. This includes a modification in which the melting temperature of the modified probe does not deviate too greatly from the melting temperature of the original probe. Said melting temperature is determined following the G(=4° C.)+C(=2° C.) rule. It is obvious to the skilled worker that it is also possible to use, in addition to the usual nucleotides A, G, C and T, modified nucleotides such as inosine, etc. The teaching of the present invention provides for such modifications, starting from the subject matter of the claims.

According to the invention, a composition comprising the nucleic acid to be detected or a part thereof is hybridized with one or more probes.

It is possible in principle to determine said nucleic acid to be detected and thus, for example, the bacterial species by hybridization with a single specific probe. However, it is also possible to hybridize said composition comprising said nucleic acid to be detected or a part thereof with more than one probe, thereby increasing the meaningfulness of the method. An accurate profile is then obtained, enabling said nucleic acid to be detected and thus, for example, the bacterial species to be determined very reliably.

The present invention furthermore relates to an apparatus for carrying out a method of the invention in the form of a rapid dry assay containing a chromatographic material comprising
a sample-receiving zone,
a separating zone having a binding region in which one or more sequence-specific nucleic acid probes are immobilized and
a liquid-absorbing zone downstream of said separating zone having a binding region, characterized in that said sequence-specific nucleic acid probes are immobilized via a polymeric linker.

The preferred embodiments of said apparatus essentially correspond to those of the method of the invention.

The sequence-specific nucleic acid probes are preferably immobilized to the membrane via a polymeric linker connected to an anchoring molecule. The polymeric linker or the polymeric linker with anchoring molecule is more than 30 nm in length. Preferably, the polymeric linker or the polymeric linker with anchoring molecule is more than 40 nm in length. Particular preference is given to the linker being polythymidine. Preference is furthermore given to the anchoring molecule being psoralen.

The preferred chromatographic material of the apparatus of the invention is nitrocellulose. Preference is furthermore given to said chromatographic material having a pore size of 4 μm or more.

In a particular embodiment of the apparatus of the invention, the sequence-specific nucleic acid probe is a nucleic acid which hybridizes under stringent hybridization conditions to a fragment from the genome of a parodontitis-associated bacterium or to the sequence complementary thereto. In said embodiment, particular preference is given to the sequence-specific nucleic acid probe being selected from any of the following sequences or being complementary to said sequences: SEQ ID No. 1-29, or comprising fragments thereof.

The present invention furthermore relates to the use of the apparatus of the invention for detecting, differentiating and characterizing nucleic acids, in particular for detecting, differentiating and characterizing parodontitis-associated bacteria.

The present invention furthermore relates to the use of the method of the invention and of the apparatus of the invention for detecting amplification products from amplification methods such as polymerase chain reaction (PCR) and nucleic acid strand-based amplification (NASBA). It is pointed out here that said amplification products may also have been produced by other nucleic acid amplification techniques such as, for example, transcriptase-mediated amplification (TMA), reverse transcriptase polymerase chain reaction (RT-PCR), Q-beta replicase amplification (β-Q-replicase) and single-strand displacement amplification (SDA).

### DESCRIPTION OF THE FIGURES

FIG. 1: Illustration of a test strip suitable for the rapid dry assay of the invention

FIG. 2: Rapid dry assay (A), positive for Actinobacillus actinomycetemcomitans, Porphyromonas gingivalis and Bacteroides forsythus, and rapid dry assay (B), positive for Actinobacillus actinomycetemcomitans. 150 μl of a test mixture were applied in drops to the application pad of the assay and incubated at room temperature for 5 min. The probes for (A) immobilized on the nitrocellulose membrane were (from top to bottom) a control (biotin-BSA) and the probes for Actinobacillus actinomycetemcomitans: 5′-PsoC6-T85-SEQ ID No.: 16, Porphyromonas gingivalis: 5′-PsoC6-T85-SEQ ID No.: 21 and Bacteroides forsythus: 5′-PsoC6-T84-SEQ ID No.: 23; i.e. all probes have been labeled in each case 5′ with psoralen and have a polythymidine linker of 85 thymidines.

(A) depicts a sample positive for all three probes, while in (B) only an Actinobacillus actinomycetemcomitans amplicon was specifically detected. Amplification was carried out using a species-specific primer pair (primer pair 5′-biotin-SEQ ID No.: 14; 5′-biotin-SEQ ID No. 4).

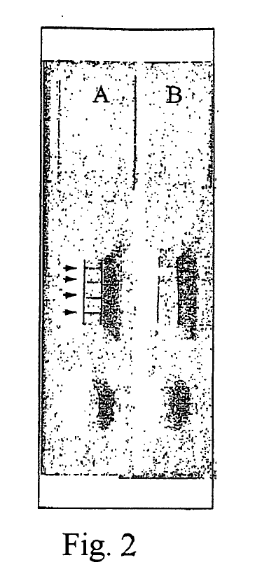

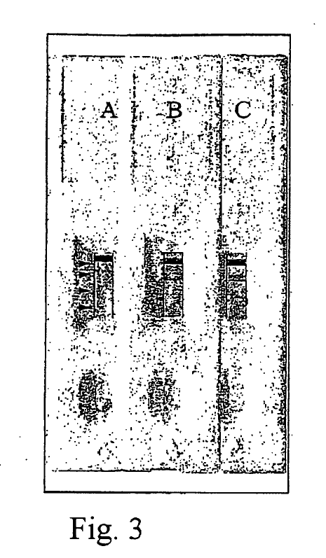

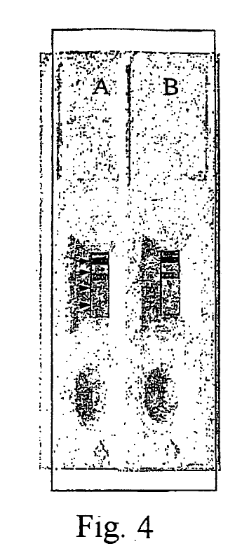

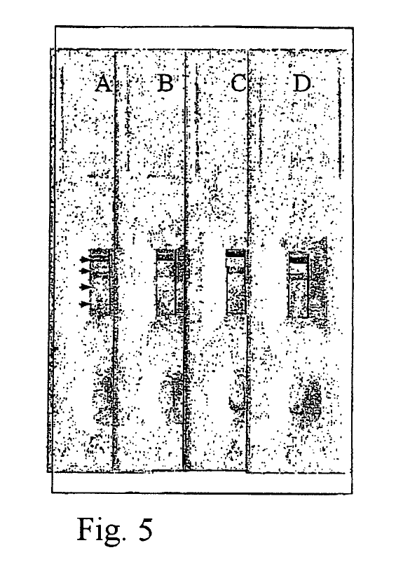

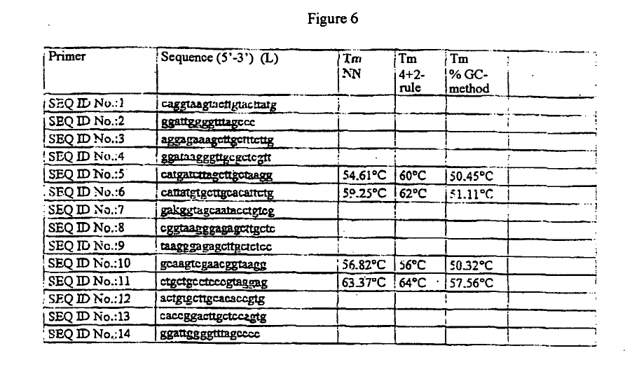

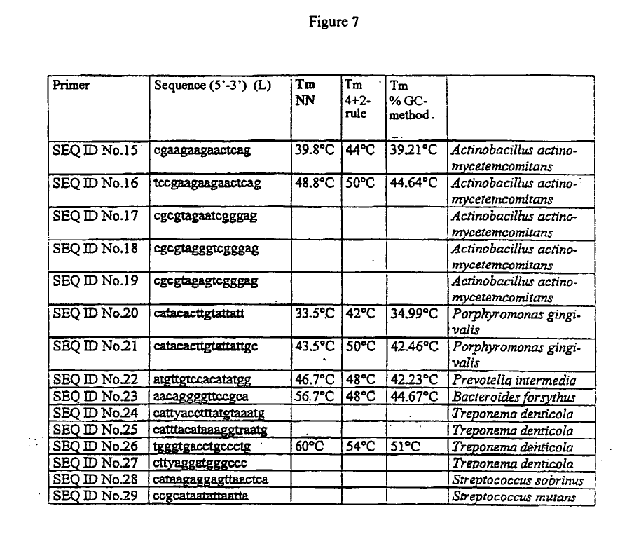

Probes used in FIG. 2:

SEQ ID No.: 16

5′PsoC6-T85- ccgaagaagaactcag:
(A. actinomycetemcomitans)

SEQ ID No.: 21

5′PsoC6-T85- catacacttgtattattgc:
(Porphyromonas gingivalis)

SEQ ID No.: 23

5′PsoC6-T85- aacaggggttccgca:
(Bacteroides forsythus)

Primers used in FIG. 2:

SEQ ID No.: 14:

5′-biotin- ggattggggtttagcccc:

SEQ ID No.: 4:

5′-biotin- ggataagggttgcgctcgtt:

FIG. 3: Influence of the linker arm length on sensitivity. Variation of the probe SEQ ID No.: 16 in the polythymidine linker: (A) no linker, (B) 20 thymidine residues and (C) 100 thymidine residues. The amplicon used was Actinobacillus actinomycetemcomitans (primer pair 5′-biotin-SEQ ID No.: 14, 5′-biotin-SEQ ID No. 4).

FIG. 4: Influence of psoralen label on sensitivity. The probe SEQ ID No. 16 having a polythymidine linker of 85 thymidine residues was immobilized without psoralen label (A) and with psoralen label (B). Actinobacillus actinomycetemcomitans (primer pair 5′-biotin-SEQ ID No.: 14, 5′-biotin-SEQ ID No. 4) was amplified in each case.

FIG. 5: Influence of mildly denaturing compounds on sensitivity. Denatured Actinobacillus actinomycetemcomitans amplicon (primer pair 5′-biotin-SEQ ID No.: 14, 5′-biotin-SEQ ID No. 4) was applied to the rapid dry assay in hybridization buffer without DMSO (A), and with 10% DMSO, 20% DMSO and 30% DMSO (B) (probe SEQ ID No. 16, psoralen-labeled and with the polymidine linker containing 85 polymidine residues).

FIG. 6 and FIG. 7: Sequences SEQ ID No. 1-29, nucleic acid probes for detecting parodontitis-associated bacteria.

### EXAMPLE 1

Detection of Parodontitis-associated Bacteria with the Aid of the Rapid Dry Assay of the Invention

For this purpose, the probes SEQ ID No. 1-29 (probes modified with psoralen at the 5′ end) are applied to a nitrocellulose membrane and bound by means of UV irradiation.

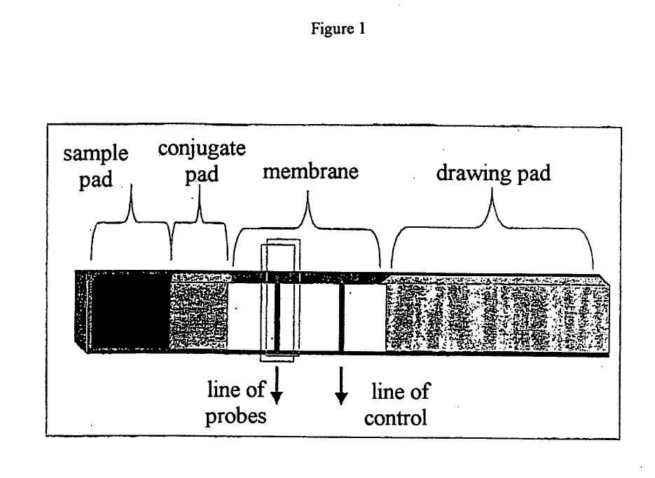

FIG. 1 depicts the design of the rapid assay. Isolation of DNA: for this purpose, bacterial material was removed from solid media, using a sterile inoculation loop, and suspended in 300 μl of 10 mM Tris/HCl pH 7.5. 1 ml was removed from liquid cultures, centrifuged in a bench centrifuge at 13 000 rpm for 5 min and, after discarding the supernatant, resuspended in 300 μl of 10 mM Tris/HCl pH 7.5. The cell suspensions obtained in this way were incubated in a Thermomixer (Eppendorf, Hamburg, Germany) at 95° C. for 15 min, sonicated in an ultrasound bath (Bandelin Electronic, Berlin, Germany) for 15 min and centrifuged in a bench centrifuge (Eppendorf, Hamburg, Germany) at 13 000 rpm for 10 min. In each case, 5 μl of the supernatant were used in the amplification reaction.

Amplification:

All primers were commercially synthesized (Interactiva, Ulm, Germany). The PCR mixture contained 1×Taq buffer (Qiagen, Hilden, Germany), in each case 1 μM primer, 200 μM DNTP (Roche, Mannheim, Germany) and 1 U of Hotstar Taq polymerase (Qiagen, Hilden, Germany). The PCR amplification was carried out on a Thermocycler PE 9600 (ABI, Weiterstadt, Germany), with 95° C. for 15 min, 10 cycles of 95° C. for 30 s and 60° C. for 2 min and 20 cycles of 95° C. for 10 s, 55° C. for 50 s and 70° C. for 30 s.

The DNA amplicon was detected using an ethidium bromide-stained agarose gel.

5′-psoralen-labeled oligonucleotide probes (Aa1, Pg2, Bf) dissolved in 3×SSC (10×SSC solution, 1.5 M NaCl, 0.15 M trisodium citrate) were applied in the form of lines to a nitrocellulose membrane AE 99 (Schleicher & Schull, Dassel, Germany) by a Probenautomat [sample dispenser] 3 “Freemode” (CAMAG, Berlin, Germany). A line of biotin-BSA (SIGMA, Munich, Germany) 1 mg/ml was sprayed on as a staining control. After the oligonucleotides had completely dried, they were fixed to the membrane in a UV crosslinker (UV Stratalinker 2400, Stratagene, La Jolla, USA) at 1200 Joule/cm². The coated membrane was glued to a vinyl back and provided with a sample pad (Grade 903 paper, Schleicher & Schüll, pretreated with 0.01 M sodium tetraborate, 1% Triton X-100, pH 7.4) and a drawing pad (Grade 470 paper, Schleicher & Schüll). The cut strips were packed into a plastic housing.

Streptavidin-conjugated gold particles, 20 nm (British Biocell, Cardiff, United Kingdom), αD524, 4.0 were utilized for detection. 3 μl of this gold particle suspension in a mixture with 20 μl of biotinated amplicon were incubated for 5 min. The amplicon was denatured by adding an NaOH solution (final concentration 200 mM). After 5 minutes of incubation, the solution was neutralized in 150 μl of running buffer comprising 250 mM phosphate buffer, pH 7.5, 50 mM NaCl, 0.1% Tween 20 and 20% DMSO (SIGMA, Munich, Germany) and the entire mixture was applied to the application zone. After approx. 5 min, the developed zones can be read.

### EXAMPLE 2

Influence of the Linker Arm Length on Sensitivity

In order to have higher sensitivity available, detection for the experiments below was carried out without conjugated particles but by way of alkaline phosphatase-catalyzed staining with NBT/BCIP. For this purpose, the strip, after the target nucleic acid had hybridized in the rapid dry assay method, was removed from the housing and washed once in 1×SSC for 1 min. By incubation in 5 g/l blocking reagent (Roche, Mannheim, Germany) in maleic acid buffer pH 7.5 (8.26 g of NaCl and 10.06 g of maleic acid in 1 l of water) containing streptavidin-alkaline phosphatase conjugate (1:5000 dilution, Dianova, Hamburg, Germany) for 15 min. After washing three times with washing buffer (3×SSC, 0.1% Tween 20), the hybridized DNA was stained by incubation in substrate buffer (274 mM Tris/Cl pH 7.5, 68.6 mM Na₃Citrate, 200 mM NaCl, 27.4 mM MgCl₂×6 H₂O) containing NBT (75 mg/ml Nitro Blue tetrazolium salt in 70% dimethylformamide) and BCIP (50 mg/ml 5-bromo-4-chloro-3-indonylphosphate-toluidinium salt in 100% dimethylformamide).

As described in example 1 the probe SEQ ID No. 16 without polythymidine linker, the same probe with polythymidine linker containing 20 thymidine residues and containing 100 thymidine residues were hybridized to the strip and developed with NBT/BCIP. The amplicon used was Actinobacillus actinomycetemcomitans (primer pair 5′-biotin-SEQ ID No.: 14; 5′-biotin-SEQ ID No. 4). A distinct sensitivity gradient is discernable, which correlates with the length of the linker (see FIG. 3). The probe whose linker is 100 thymidine residues in length displays the highest sensitivity.

### EXAMPLE 3

Influence of Psoralen Label on Sensitivity

As described in example 1, the probe SEQ ID No. 16 having a polythymidine linker of 100 thymidine residues with 5′-psoralen label and the same probe without 5′-psoralen label were hybridized to the strip and developed with NBT/BCIP. The amplicon used was Actinobacillus actinomycetemcomitans (primer pair 5′-biotin-SEQ ID No.: 14, 5′-biotin-SEQ ID No. 4). A distinct sensitivity gradient is discernable which correlates with the presence of the psoralen label (FIG. 4). Psoralen-labeled probes are more sensitive.

### EXAMPLE 4

Influence of Mildly Denaturing Compounds on Sensitivity:

As described in example 1, the probe SEQ ID No. 16 having a polythymidine linker of 100 thymidine residues and the same probe with 5′-psoralen label were hybridized to the strip and developed with NBT/BCIP. Denatured Actinobacillus actinomycetemcomitans amplicon (primer pair 5′-biotin-SEQ ID No.: 14, 5′-biotin-SEQ ID No. 4) was applied in hybridization buffer without DMSO (A), or with 10% DMSO, 20% DMSO and 30% DMSO (B) to the rapid dry assay; i.e. the running buffer contains increasing concentrations of DMSO of 0, 10, 20 and 30%.

The assay is more sensitive at DMSO concentrations of more than 10% (FIG. 5).

1. A method for detecting, differentiating and characterizing nucleic acids in the form of a rapid dry assay; said rapid dry assay containing a chromatographic material comprising
a sample-receiving zone,
a separating zone having a binding region in which one or more sequence-specific nucleic acid probes are immobilized and
a liquid-absorbing zone downstream of said separating zone having a binding region,
characterized in that
said sequence-specific nucleic acid probes are immobilized via a polymeric linker, and further characterized in that said method comprises the following steps:
i) in the case of double-stranded nucleic acids, denaturing and then neutralizing the nucleic acid to be detected,
ii) applying the nucleic acid to be detected to said sample-receiving zone in a running buffer containing mildly denaturing agents,
iii) the nucleic acid to be detected moving from said sample-receiving zone in the direction of said liquid-absorbing zone,
iv) the nucleic acid to be detected being contacted with, and hybridizing to, said sequence-specific nucleic acid probe in said binding region of the separating section,
v) detecting the nucleic acid to be detected or hybridization of the nucleic acid to be detected to said sequence-specific nucleic acid probe via a label attached to the nucleic acid to be detected or via detecting a label of the nucleic acid double strand.

2. The method as claimed in claim 1, characterized in that the sequence-specific nucleic acid probes are immobilized to the membrane via a polymeric linker connected to an anchoring molecule.

3. The method as claimed in claim 1, characterized in that the polymeric linker or the polymeric linker with anchoring molecule is more than 30 nm in length.

4. The method as claimed in claim 3, characterized in that the polymeric linker or the polymeric linker with anchoring molecule is more than 40 nm in length.

5. The method as claimed in claim 1, characterized in that the linker is polythymidine.

6. The method as claimed in, claim 2 characterized in that the anchoring molecule is psoralen.

7. The method as claimed in claim 1, characterized in that the nucleic acid to be detected is labeled with biotin or fluorescein.

8. The method as claimed in claim 1, characterized in that the chromatographic material is nitrocellulose.

9. The method as claimed in claim 1, characterized in that denaturation according to method step i) is carried out with NaOH and neutralization according to method step i) is carried out with a phosphate buffer.

10. The method as claimed in claim 1, characterized in that the running buffer according to method step ii) is phosphate buffer and the mildly denaturing agent contained in said running buffer is one or more of the following: formamide, DMSO, urea.

11. The method as claimed in claim 1, characterized in that the nucleic acid to be detected is a fragment from the genome of a parodontitis-associated bacterium or is complementary thereto.

12. The method as claimed in claim 10, characterized in that the sequence-specific nucleic acid probe is a nucleic acid having a species-specific sequence which hybridizes to the nucleic acid to be detected under stringent hybridization conditions.

13. The method as claimed in claim 10, characterized in that the sequence-specific nucleic acid probe is selected from any of the following sequences or is complementary to these sequences:
SEQ ID No. 1-29 or comprises fragments thereof.

14. An apparatus for carrying out a method as claimed in claim 1 in the form of a rapid dry assay containing a chromatographic material comprising
a sample-receiving zone,
a separating zone having a binding region in which one or more sequence-specific nucleic acid probes are immobilized and
a liquid-absorbing zone downstream of said separating zone having a binding region,
characterized in that
said sequence-specific nucleic acid probes are immobilized via a polymeric linker.

15. The apparatus as claimed in claim 14, characterized in that the sequence-specific nucleic acid probes are immobilized to the membrane via a polymeric linker connected to an anchoring molecule.

16. The apparatus as claimed in claim 14, characterized in that the polymeric linker or the polymeric linker with anchoring molecule is more than 30 nm in length.

17. The apparatus as claimed in claim 14, characterized in that the polymeric linker or the polymeric linker with anchoring molecule is more than 40 nm in length.

18. The apparatus as claimed in claim 14, characterized in that the linker is polythymidine.

19. The apparatus as claimed in claim 14, characterized in that the anchoring molecule is psoralen.

20. The apparatus as claimed in claim 14, characterized in that the chromatographic material is nitrocellulose.

21. The apparatus as claimed in claim 14, characterized in that the chromatographic material has a pore size of 4 μm or more.

22. The apparatus as claimed in claim 14, characterized in that the sequence-specific nucleic acid probe is a nucleic acid which hybridizes under stringent hybridization conditions to a fragment from the genome of a paradontitis-associated bacterium or to the sequence complementary thereto.

23. The apparatus as claimed in claim 14, characterized in that the sequence-specific nucleic acid probe is selected from any of the following sequences or is complementary to these sequences: SEQ ID No. 1-29 or comprises fragments thereof.

24. The use of an apparatus as claimed in claim 14 for detecting, differentiating and characterizing nucleic acids.

25. The use of an apparatus as claimed in claim 14 for detecting, differentiating and characterizing paradontitis-associated bacteria.
